(19) United States
(12) Patent Application Publication
(10) Pub. No.: US 2008/0221566 A1
(43) Pub. Date: Sep. 11, 2008

(54) Method and apparatus for detecting and achieving closure of patent foramen ovale

(76) Inventor: Subramaniam C. Krishnan, Irvine, CA (US)

Correspondence Address: DINSMORE & SHOHL, LLP, 1900 CHEMED CENTER, 255 EAST FIFTH STREET, CINCINNATI, OH, 45202, US

(21) Appl. No.: 11/605,491

(22) Filed: Nov. 29, 2006

Related U.S. Application Data
(60) Provisional application No. 60/740,512, filed on Nov. 29, 2005.

(51) Int. Cl. A61B 18/14; A61B 19/00; A61B 17/22

(52) U.S. Cl. 606/41; 606/130; 606/159

(57) ABSTRACT

A method for detecting and closing the patent foramen ovale including the steps of locating a His bundle, plane of the interatrial septum, and coronary sinus ostium in a patient; identifying a fossa ovalis on the basis of one or more predetermined distances between the fossa ovalis and the His bundle, the plane of the interatrial septum, and the coronary sinus ostium; locating a patent foramen ovale by probing the junction between the fossa ovalis and a limbus of the fossa ovalis; and causing injury to the surfaces of at least one of a septum primum and a septum secundum within the patent foramen ovale. Another method includes the steps of locating a tunnel of a patent foramen ovale by probing the junction between a fossa ovalis and a limbus of the fossa ovalis and causing injury to the surfaces of at least one of a septum primum and a septum secundum within the tunnel of the patent foramen ovale by applying energy to at least one of the septum primum and the septum secundum. Apparatuses to perform these methods are also provided.

### RELATED APPLICATION

The present application claims priority under 35 U.S.C. § 119 to U.S. Application Ser. No. 60/740,512 filed Nov. 29, 2005, and which is herein incorporated by reference.

### TECHNICAL FIELD

The present invention relates to methods and apparatuses for detecting and closing the patent foramen ovale. Particularly, the present invention relates to locating and/or applying energy within the patent foramen ovale to provide closure.

### BACKGROUND OF THE INVENTION

Recently, the congenital cardiac anomaly of patent foramen ovale (PFO) has been receiving significant attention. It may be a risk factor for diseases and clinical syndromes such as embolic strokes, embolic myocardial infarctions, decompression sickness as well as migraine headaches with associated visual aura (see, e.g., Wu L A, Malouf J F, Dearani et al. Arch Intern Med 2004; 164: 950-956; and Kerut E K, Norfleet W T, Plotnick G D, et al. J AM Coll Cardiol 2001; 38: 613-623; and Torti S R, Billinger M, Schwerzmann M et al. Eur Heart J 2004; 25: 1014-1020). It is felt that in a significant number of patients with a stroke and no risk factor, the stroke may have happened because a blood clot from the venous circulation flowed across the PFO into the arterial circulation and into the brain (giving rise to a cryptogenic stroke) or the heart, rather than the lungs. Some experts feel that patients with PFO who have had a cryptogenic stroke, have a 4% risk per year of having another stroke. Recently, several independent observations have been noted of an association between PFO closure and a substantial reduction in migraine headaches (Tsimikas S. J Am Coll Cardiol 2005; 45: 496-498). With 12% of the population suffering from migraine headaches, these observations have generated much interest in using different methodologies to achieve closure of PFOs.

The foramen ovale is necessary for blood flow across the fetal atrium. Beginning at four weeks of pregnancy, the primordial single atrium divides into right and left sides by formation of two septa: the septum primum and septum secundum. These two structures overlap, but are not fused in fetal life. The opening present between the two septa (due to the absence of fusion of the two structures) is the foramen ovale. The septum primum forms a flap-like valve over the foramen ovale, which typically closes by fusing with the growing septum secundum after birth. In utero, as oxygenated blood flows from the inferior vena cava and enters the right atrium, it crosses the patent foramen ovale and becomes the systemic circulation. After birth, right heart and pulmonary pressures drop as pulmonary arterioles open in reaction to oxygen filling the alveolus. The left atrial pressures also rise as the amount of blood returning from the lungs increases. These mechanisms cause a flap closure against the septum secundum. By age two, the fusion is complete in about 75% of individuals, but patency remains in the other 25%. In these individuals, the patent foramen ovale is a residual, oblique, slit-like defect resembling a tunnel.

Initial methods that were developed to close PFOs consisted of surgical closure of the slit-like tunnel. However, performing open heart surgery purely to close a PFO that is very often of doubtful significance is difficult to justify.

More recently, a number of devices for closing PFOs percutaneously have been developed, offering a less invasive alternative to open heart surgery. Most of these are similar in design to devices developed to close atrial septal defects and are typically a “clamshell” or a “double umbrella” which deploy a device made of biocompatible metal or fabric on both sides of the septum which then “sandwiches” the overlapping septum primus and secundum. There then occurs a “healing” process accompanied by endothelialization of the device. Several such devices have been developed including the “cardioSEAL” device from NMT Medical company (Boston, Mass.), the Amplatzer device made by AGA corporation (Golden valley MN), and others. Several problems can be associated with this approach, as outlined below.

Complications of device implantation occur in 6-10% of patients and include device embolization, fracture of the device, incomplete closure, air embolism, vascular complications, device-related thrombi, cardiac tamponade, hemorrhage requiring blood transfusion and urgent surgical intervention, pulmonary embolism and even death. See, e.g., Windecker S, Wahl A, Nedeltchev K, et al. J Am Coll Cardiol 2004; 44: 750-758; Khairy P, O'Donnell C P, Landzberg M J. Ann Intern Med 2003; 139: 753-760; and Krumsdorf U, Ostermayer S, Billinger K, et al. J Am Coll Cardiol 2004; 43: 302-309.

Methods for the transcatheter closure of PFOs without the use of implantable devices have also been developed. These methods generally can be divided into two approaches:

(a) Injury & endothelial denudation of apposed tissues with delayed healing and fibrosis: In this approach, injury and endothelial denudation is created along the apposed/adjoining surfaces of the septum primum and septum secundum (i.e. within the tunnel of the PFO), using mechanical measures or with application of RF energy. It is hypothesized that the healing process results in the development of adhesions between the foramen primum and secundum, resulting in closure of the patent foramen ovale. This method assumes that demonstrating acute closure is not important and does not reflect the likelihood of long term success.

(b) Tissue fusion or tissue welding: This concept emphasizes bringing tissues together and applying energy to the tissues. Using this principle, it has been hypothesized that acute closure of the PFO will result in a substantial manner. The term substantial has been characterized by the formation of a “stable tissue bridge” between the septum primum and secundum, and this bridge will purportedly withstand physiological pressures. The acuity of the closure supposedly distinguishes this concept from that of fusion due to healing and scarring.

### SUMMARY OF THE INVENTION

In accordance with one embodiment of the present invention, a method of closing a patent foramen ovale includes the steps of locating a His bundle, plane of the interatrial septum, and coronary sinus ostium in a patient; identifying a fossa ovalis on the basis of one or more predetermined distances between the fossa ovalis and the His bundle, the plane of the interatrial septum, and the coronary sinus ostium; locating a patent foramen ovale by probing the junction between the fossa ovalis and a limbus of the fossa ovalis; and causing injury to the surfaces of at least one of a septum primum and a septum secundum within the patent foramen ovale.

In accordance with another embodiment of the present invention, a method of closing a patent foramen ovale in a patient, including the steps of locating a tunnel of a patent foramen ovale by probing the junction between a fossa ovalis and a limbus of the fossa ovalis; and causing injury to the surfaces of at least one of a septum primum and a septum secundum within the tunnel of the patent foramen ovale by applying energy to at least one of the septum primum and the septum secundum.

In accordance with yet another embodiment of the present invention, an apparatus for closing a patent foramen ovale in a patient, including a catheter and an abrading surface. The catheter has one or more electrodes at the distal end. The abrading surface is located proximate to the distal end of the catheter.

### BRIEF DESCRIPTION OF THE DRAWINGS

While the specification concludes with claims particularly pointing out and distinctly claiming the present invention, it is believed that the same will be better understood from the following description taken in conjunction with the accompanying drawings in which:

FIG. 1 depicts an embodiment of a left atrial diverticulum;

FIG. 2 illustrates another embodiment of a left atrial diverticulum;

FIG. 3 illustrates an embodiment of a right atrial diverticulum showing adhesion in a heart occurring in a tunnel providing a diverticulum accessible from the right atrium;

FIG. 4 illustrates the level of adhesion between the septum primum and septum secundum resulting in diverticulum accessible from the right atrium;

FIG. 5 depicts the closure of a PFO by use of a catheter;

FIG. 6 illustrates an embodiment of using an electroanatomical navigation system to image and size PFOs;

FIG. 7A illustrates anatomic variation for the location of a PFO;

FIG. 7B depicts an embodiment of using a virtual fossa to probe for a PFO;

FIG. 8 illustrates an embodiment measuring impedance changes as a factor to determine amount of energy applied in a PFO;

FIG. 9 depicts an embodiment of an apparatus to apply mechanical abrasion once the PFO tunnel has been located; and

FIG. 10 illustrates an embodiment of an apparatus to apply mechanical abrasion once the PFO tunnel has been located.

### DETAILED DESCRIPTION OF EMBODIMENTS

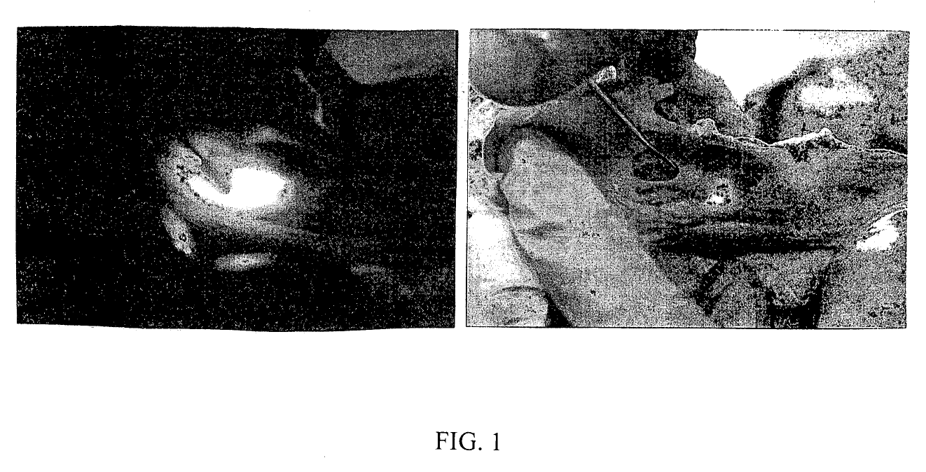

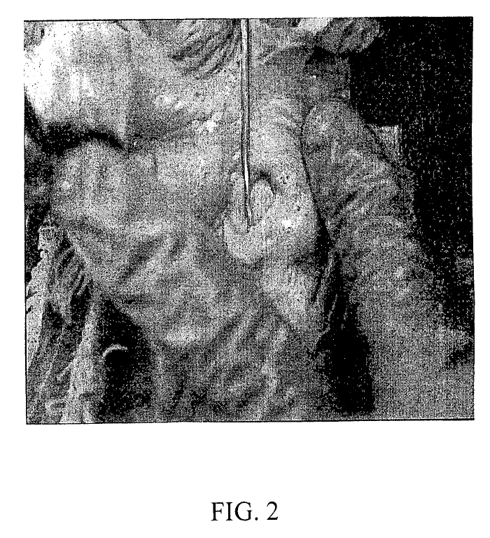

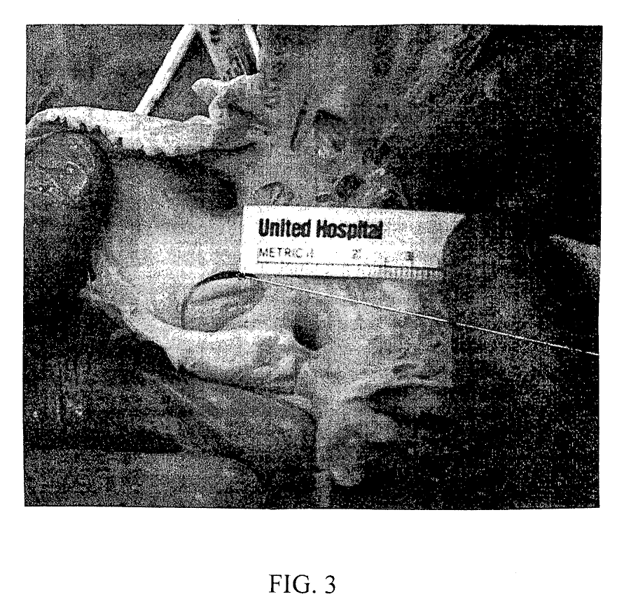

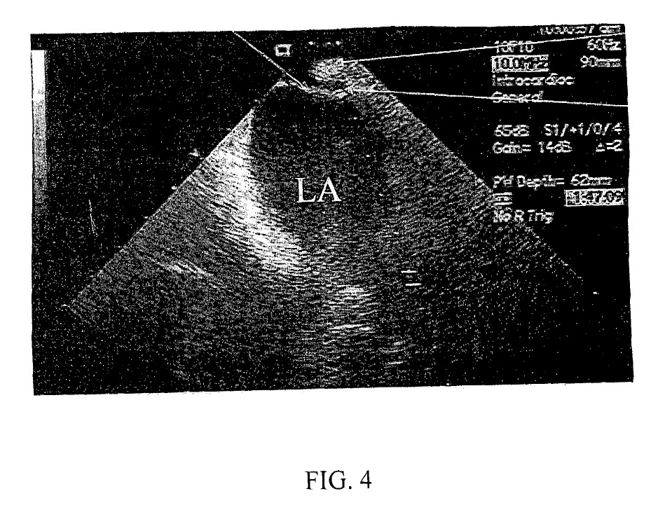

Embodiments of the present invention and its operation are hereinafter described in detail in connection with the views and examples of FIGS. 1-10, wherein like numbers indicate the same or corresponding elements throughout the views. Applicant has discovered that if a patent foramen ovale is used as a route to access the left atrium for electrophysiology procedures, this often results in closure of the PFO. In fact, a correlation has been noted between the duration of the procedure and the likelihood of closure—suggesting that greater trauma is more likely to result in closure of the PFO. Autopsy observations (made by the Applicant) also show that spontaneous closure of a PFO often occurs only at certain points/locations between the septum primum and septum secundum (often giving rise to pouches or diverticula) rather than along the entire length of the overlap of the septum primum and the septum secundum. (see FIGS. 1-4). This suggests that creating injury between the two structures may serve to create adhesions between the two structures and reproduce the natural history of PFO closure. Injury creation to adjoining surfaces can be accomplished to provide closure, however, acute closure is not necessary. The present methodologies and apparatuses can provide less injury to the surfaces, thus when energy is applied to cause the injury, less energy application is necessary, which in turn diminishes the likelihood of damaging adjoining structures.

Based upon these findings, Applicant has developed apparatus and methods for achieving PFO closure by creating injury and endothelial denudation along apposing and overlapping surfaces of the septum secundum and septum primum. Following injury and denudation, healing results in adhesion between the two surfaces and hence closure of the PFO. In addition, the apparatus and methods may be configured such that adhesion formation occurs across the entire area of overlap, rather than select portions of overlap between the septum secundum and septum primum.

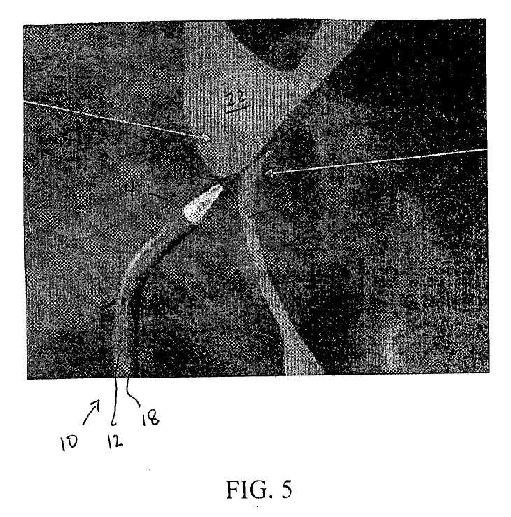

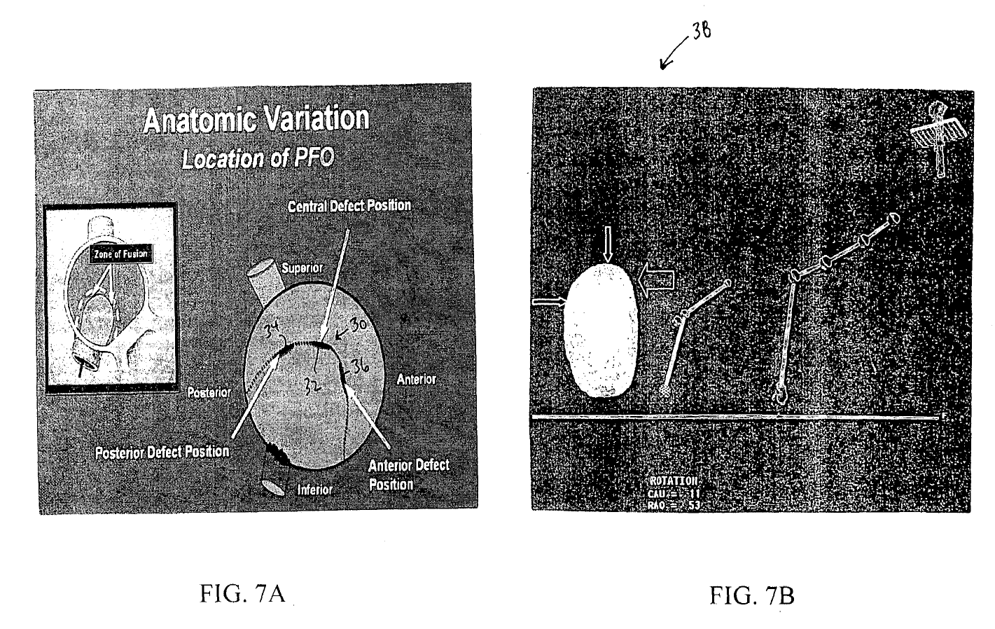

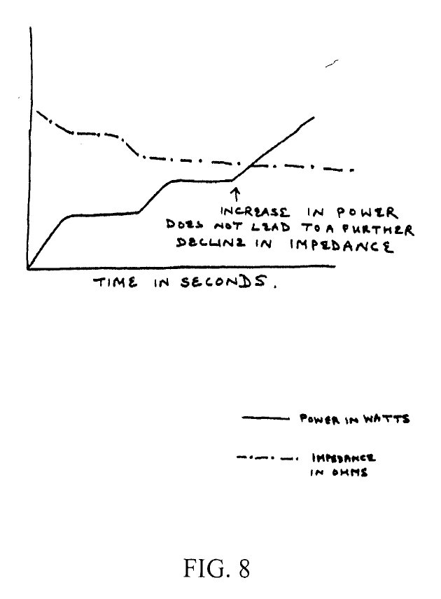

FIG. 5 depicts one exemplary embodiment of an apparatus 10 for performing the methods of the present invention. This apparatus includes a catheter 12 which may be similar, or even identical to, the catheter 12 shown in Applicant's pending U.S. patent application Ser. No. 10/648,844, filed Aug. 25, 2003, which is incorporated herein by way of reference (hereinafter “the '844 application”). Thus, the catheter 12 shown in FIG. 5 may include a tapered distal end 14 and one or more electrodes 16 at the distal end 14 of the catheter 12 (see FIGS. 7 and 8 of the '844 application). FIG. 5 further illustrates the relationship between the septum primum 20 and the septum secundum 22.

It should be noted, however, that the distal end 14 of the catheter 12 of the present invention need not be tapered or conical. As further shown and described in the '844 application, the apparatus shown in FIG. 5 herein may further include a hollow sheath (not shown) in which the catheter 12 is positioned during use. The catheter 12 of FIG. 5 may be advanced along a guidewire 18, as described in the '844 application. It should be noted that an exemplary guidewire 18 is depicted in FIG. 5 herein. As further described herein, the catheter 12 of the present invention may be steerable or deflectable in the same manner as conventional catheters used to perform catheter ablation of cardiac arrhythmias.

In order to use the catheter 12 of FIG. 5 or those described in the '844 application, the catheter 12 is inserted into the patient in the manner described in the '844 application. Thereafter, the distal end 14 of the catheter 12 is dragged down along the septal surface of the atrium. The fossa ovalis is then identified by one or more of the following features, as described in the '844 application: i) a diminished unipolar and/or bipolar voltage of the electrogram recorded from the tip of the apparatus; ii) a diminished slew rate; iii) an elevated pacing threshold; and/or iv) diminished impedance. The fossa ovalis can also be identified based on distances between the fossa ovalis and the coronary sinus ostium on the septal plane—creation of a virtual fossa ovalis.

Once the fossa ovalis has been located, the patent foramen ovale may be readily identified and located. For example, the tip of the catheter may be used to probe the junction between the fossa ovalis and the limbus. The probing of the junction between the fossa ovalis and the limbus is done in a systematic manner (like the different segments of the circumference of a clock). The areas that have been probed can be marked with the electroanatomical navigation system. If the tip passes into the left atrium, a PFO is present. Once identified, the catheter may be used to further probe the junction between the fossa ovalis and limbus in order to determine the dimensions of the PFO. By way of further example, fluoroscopy may be used to observe the tip of the catheter entering the left atrium. In the left anterior oblique projection, as the junction between the fossa ovalis and the limbus is probed, it will be seen if the catheter crosses into the left atrium. The electroanatomical navigation system can also be used to measure the dimensions of the fossa ovalis.

The apparatus 10 shown in FIG. 5 and shown and described in the '844 application may be used to close a PFO by applying energy from one or more energy emitters (e.g., electrodes 16) located at the distal end 14 of the catheter 12 within the “tunnel” 24 located between the overlapping portions of the septum primum 20 and the septum secundum 22. This will induce endothelial denudation and thus lead to adhesions between the two adjoining surfaces which leads to PFO closure. In this particular embodiment, the energy applied by the catheter 12 may comprise RF current (unipolar or bipolar). However, alternatively, or in addition thereto, the catheter 12 (particularly, the distal end 14 thereof) may be configured to emit laser energy, microwaves or high intensity ultrasound in order to induce endothelial denudation. When unipolar RF current is employed, a single electrode 16 may be provided at the distal end 14 of the catheter 12. However, as further discussed herein, one or more additional electrodes may be incorporated into the distal end 14 of the catheter 12 in order to allow integration of the catheter 12 into an electroanatomical mapping/navigation system. In addition, one or more additional features, such as magnetic field sensors, may be provided in the distal end 14 of the catheter 12 for incorporation into an electroanatomical mapping system such as the CARTO system from Biosense-Webster.

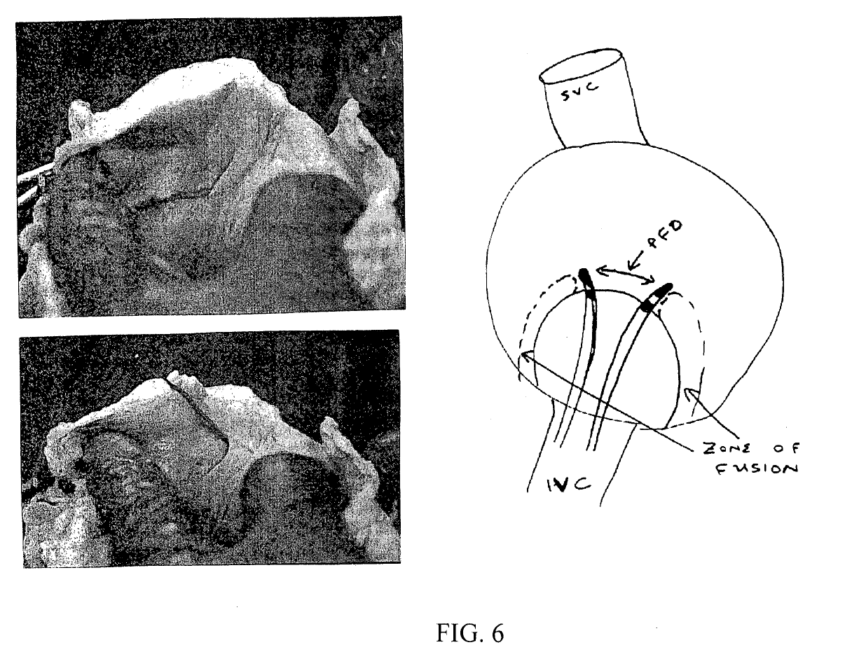

The apparatuses and methods described in the '844 application for locating the fossa ovalis (i.e., the location of a PFO) may also be used to not only locate the fossa ovalis but also to measure the dimensions of the PFO. Alternatively, or in addition thereto, the apparatus and methods described in Applicant's U.S. Provisional Patent Application No. 60/658,111, filed Mar. 3, 2005 (“the '111 application”, which is incorporated herein by way of reference) may be used to locate the fossa ovalis, including the creation of a “virtual fossa ovalis” (as described in the '111 application). In particular, once the fossa ovalis is located, the tip of the catheter may be used to probe between the limbus and the fossa ovalis in order to not only locate the PFO but also measure its size. FIG. 6 herein depicts a technique for locating, imaging and sizing a PFO by using a catheter to probe between the limbus and the fossa ovalis. As shown in FIG. 6, the catheter enters the tunnel of the PFO to provide fusion of the septum primum and the septum secundum.

FIGS. 7A and 7B depict a method of locating a PFO 30, including its boundaries, by the creation of a virtual fossa ovalis in accordance with the '111 application. Thus, as shown in FIG. 7A, a PFO may be located at central 32, posterior 34 and/or anterior 36 defect positions. By using an electroanatomical mapping system 38 (e.g., the CARTO system from Biosense-Webster, the LOCALISA system from Medtronic, the NAVX system from St. Jude Medical, or the RPM system from Boston Scientific) to locate the fossa ovalis, the location and dimensions of the PFO 30 can be readily determined and a 3-dimensional reconstruction of the PFO tunnel can be displayed on a display device associated with the mapping system. The PFO may be located by probing the fossa ovalis, especially the junction between the fossa ovalis and the limbus of the fossa ovalis, and it may be observed (e.g., using fluoroscopy or the mapping system itself) if the catheter passes into the left atrium. The electroanatomical navigation system may be configured to create a 3-dimensional PFO which is displayed on the display screen associated with the mapping system. This 3-dimensional reconstruction of the PFO will identify the location and size of the PFO, and any visible indicia may be displayed to the user as the PFO.

This 3-dimensional reconstruction of the PFO may be determined by software included in the electroanatomical mapping system. By way of example, the catheter may be used to probe the junction between the fossa ovalis and the limbus, and the software may be configured to detect whenever the tip of the catheter passes into the left atrium. The PFO may then be reconstructed and displayed on a display screen associated with the mapping system, such as in conjunction with the virtual fossa ovalis determined and displayed in accordance with the '111 application.

Once the PFO has been located, the catheter, specifically the distal end thereof, may be used to apply energy (e.g., RF current, laser energy, microwaves, and/or high intensity ultrasound). In particular, the distal end of the catheter may be placed in the PFO tunnel and energy applied to both walls of the PFO along the entire length of overlap of the two structures (i.e., the septum primum and secundum). Energy application may be continued for a predetermined amount of time and/or quantity of energy. Alternatively, energy applications may be titrated up until endpoints are detected or determined (as further described herein).

Due to low blood flow in the PFO tunnel, it may be difficult to deliver sufficient power during energy application. The low blood flow will result in the overheating of catheter, particularly when delivering RF energy to the tissue. In order to avoid overheating of the catheter, the electrode(s) at the distal end of the catheter may be enlarged over that commonly used in anatomical mapping and the like. For example, the electrode(s) may be enlarged so as to have a length of between about 8 and about 10 mm, rather than the 4 mm length used conventionally.

Alternatively, or in addition thereto, the distal end of the catheter may be actively cooled, such as through irrigation (closed irrigation system or an open irrigation system). Such active cooling may be provided, for example, by a fluid which is circulated in the interior of the distal end of the catheter, particularly a cooled fluid. In an open irrigation system, one or more passageways may be provided at or adjacent to the distal end of the catheter such that a fluid, particularly a cooled fluid such as saline, is urged into the interior of the catheter and exits the catheter from the passageways (or openings) provided at or adjacent to the distal end thereof. Such fluid emitted from these passageways will act to cool the distal end of the catheter.

In some patients, it may be desirable to ensure that energy application is gated to blood flow—i.e., energy is only applied to the tissue during systole or diastole. This may be particularly important when energy is applied adjacent to the aorta such that the “heat sink” effect is used to minimize the likelihood of damage to the aorta. This may be especially necessary if the septum secundum is shorter than normal, increasing the likelihood of contact of the energy delivery catheter with the aortic wall. This technique is further described in Applicant's U.S. patent application Ser. No. 11/259,881, entitled Methods and Systems for Gated or Pulsed Application of Ablative Energy in the Treatment of Cardiac Disorders, filed Oct. 27, 2005, which is incorporated herein by way of reference.

The use of the apparatus and methods of the '844 and/or '111 applications is also advantageous in that the application of energy in the PFO tunnel can be recorded. In fact, when an anatomical mapping system is employed, the application of energy within the PFO tunnel can be recorded three dimensionally. This will allow the practitioner to maintain a diary or catalogue, 3-dimensionally in space, of where in the PFO tunnel the energy applications have been performed. This information can be used to ensure that energy applications have been performed throughout the PFO tunnel and along the apposing walls and thus maximize chances of PFO closure.

Changes in the electrical properties of the tissue within the PFO tunnel to which energy is applied may also be monitored. Changes in the electrical properties of the tissue to which the energy is applied can be used to gauge the amount of damage to the tissue in order to determine the endpoint of the procedure. A reduction of bipolar and/or unipolar electrogram amplitudes, lower slew rate, development of pronounced ST segment elevation in the unipolar electrogram, and/or changes in impedance will indicate the presence of adequate tissue injury. For example, a decrease of the electrogram signal amplitude of at least about 75% of its baseline value may signify an endpoint for each location of energy application. Changes in impedance may be monitored as the energy is applied to the tissue. Changes in the amplitude of the electrogram signal, on the other hand, may be periodically measured after each energy application to the tissue. Thus, measuring the size of electrical signals after energy application or mechanical abrasion can be used to determine/confirm the presence of injury to the tissue.

By way of example, impedance changes in the tissue may be used to determine the endpoint of energy application within the PFO tunnel. During catheter ablation (i.e., the application of RF energy), thermal injury results in the death of heart cells (this process is called coagulation necrosis). Immediately after cell death, fluid leaks out from the intra to the extracellular compartment. Due to the increased amounts of fluid present, there will be a lower resistance (termed impedance) to current flow across the tissue. This principle can be used to determine whether energy application at a particularly site has achieved the maximal necrosis possible, using the following exemplary algorithm. Catheter ablation is begun, resulting in a decline in impedance of a certain amount, after which a plateau phase is reached. Further increases in energy/power will result in necrosis of additional myocardium with leakage of more fluid into the extracellular space with a consequent further decline in impedance (due to increased amounts of fluid in the interstitial space). The same process, i.e. stepping up of the energy power, is repeated until it is observed that further increases in power do not result in further decrements in impedance. This indicates that there is no further available myocardium within the circuit where further energy application would result in additional necrosis, i.e. the maximal possible necrosis has been achieved. This concept is graphically illustrated in FIG. 8.

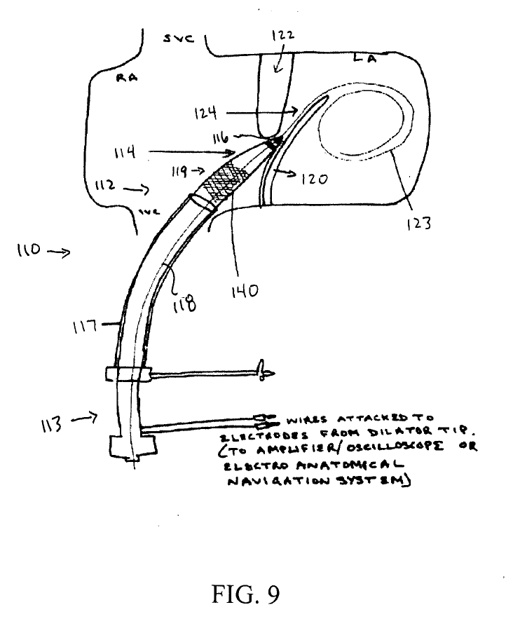

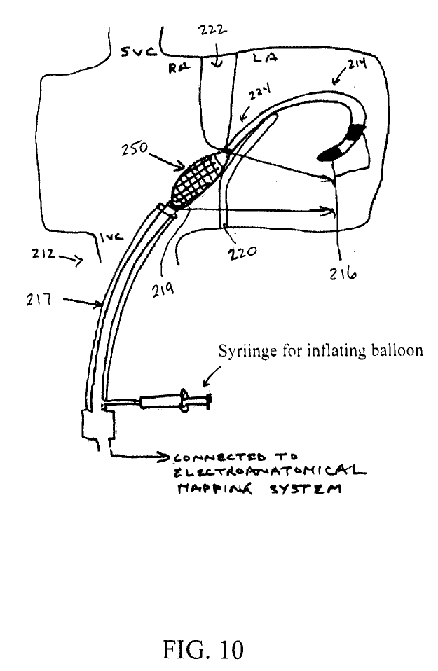

It is also contemplated that mechanical abrasion may be used in place of, or in conjunction with, the application of energy to the tissue surfaces within the PFO tunnel. FIGS. 9 and 10 depict exemplary embodiments of devices which may be used to perform mechanical abrasion of the tissue surfaces. As shown in FIGS. 9 and 10, and as further described herein, one or more electrodes 116 may be provided on the apparatus shown. These electrodes 116 may be used in conjunction with an electroanatomical mapping/navigation system, as previously described herein, in order to identify and locate the PFO and also to direct positioning of the apparatus 110 for purposes of mechanical abrasion. Of course it is also contemplated that the apparatus 110 of the present invention may be used to apply energy to the tissue surfaces within the PFO tunnel 124 as well as perform mechanical abrasion. Thus, the electrodes 116 shown in the apparatus of FIGS. 9 and 10 may be configured for applying energy to apposing surfaces of the septum primum 120 and septum secundum 122, in the manner described previously herein.

In the embodiment of FIG. 9, a catheter (or dilator) 112 similar to that described previously may be employed. Thus, one or more electrodes may be provided at the distal end 114 of the catheter 112. These electrodes 116 may be used to allow the dilator 112 to be incorporated into an electroanatomical mapping/navigation system, as previously described. Alternatively, or in addition thereto, these electrodes 116 may be used as energy emitters which cause injury to the tissue in the PFO tunnel 124, as previously described. In addition, a guidewire 118 may be used to direct the catheter 112 to the proper location. In the embodiment shown in FIG. 9, however, this guidewire 118 may be provided with a preformed spring coil. Once the guidewire 118 is directed into the left atrium, with the tip of the dilator 112 positioned within or adjacent the PFO, the end of the guidewire 118 will coil within the left atrium. In this manner, the coiled end of the guidewire 118 will prevent the catheter 112 from being advanced too far into the left atrium such that injury to the interior wall of the left atrium might occur (particularly during mechanical abrasion).

As further described below, the exterior surface of the catheter 112 proximate to the distal end 114 thereof may be roughened or otherwise provided with a surface suitable for the mechanical abrasion of the apposing surfaces within the PFO tunnel 124.

In the embodiment of FIG. 9, dilators 112 of different sizes (about 7 French to about 16 French outer diameter) may be used, depending on the size of the PFO. The dilator 112 may be made of a substance similar to dilators 112 used for transseptal puncture (e.g., PVC, polystyrene, polypropylene, polyethylene, etc.). The dilator 112 should be stiff enough so that pushing the proximal end 113 of the sheath 117 will translate into forward movement of distal end 114 of the dilator 112. At the same time, the dilator 112 should be soft/malleable enough to bend so that it may be advanced over a wire.

An abrading surface region 119 is provided on the exterior surface of the catheter 112, proximate and adjacent to the distal end 114 of the catheter 112. The abrading surface region 119 may be located between about 1 and about 4 cm (e.g., about 2-3 cm) from the tip of the catheter 112. The abrading surface region 119 may extend a distance of between about 1 and about 3 cm in length. These distances and lengths are merely exemplary of one contemplated embodiment. One or more electrodes 116 may also be provided at one or both ends of the abrading surface region 119 in order to mark the proximal 113 and distal 114 ends thereof. When an electroanatomical mapping system is employed, these electrodes 116 may be used to guide the positioning of the abrading surface region 119 during use (i.e., so that the user will know when the abrading surface region 119 is positioned within the PFO).

Like the apparatus described previously herein, as well as that shown in the '844 and '111 applications, the apparatus shown in FIGS. 9 and 10 include an outer sheath 117 through which the catheter (or dilator) 112 extends. In the embodiment of FIGS. 9 and 10, the abrading surface region 119 of the catheter/dilator 112 should be positioned within the outer sheath 117 as the apparatus 110 is inserted into the patient. In this manner, the outer sheath 117 will prevent the abrading surface region 119 from injuring the patient during insertion. Once the outer sheath 117 has been advanced into the heart adjacent the PFO, the abrading surface region 119 may be exposed by urging the catheter/dilator 112 further into the outer sheath 117. In order to accommodate the abrading surface region 119 and allow it to be exposed for purposes of mechanical abrasion, the outer sheath 117 may be at least 6 cm shorter than the length of the catheter/dilator 112. In conventional catheter assemblies such as those used to perform catheter ablation of cardiac arrhythmias, the outer sheath is typically 3-4 cm shorter than the catheter positioned within the outer sheath 117.

In the embodiment shown in FIG. 9, the abrading surface region 119 comprises rough gradations 140 etched into the surface of the catheter 112. Of course various other types of abrading surface regions 119 may be provided. The grit size of the abrading surface region 119 should be fine enough so that upon rotating the catheter 112 or moving the catheter 112 back and forth within the PFO tunnel 124 it will create the necessary injury and endothelial denudation, but will not “break off” tissue pieces/fragments of larger size that may embolize to the systemic circulation.

During use, once the PFO has been located, the abrading surface region 119 is positioned within the PFO and the catheter 112 rotated and/or moved back and forth against the apposing surfaces of the septum primum 120 and septum secundum 122 in order to “file” (i.e., abrade) the surface of the tissue. It is also contemplated that moving the dilator/abrading apparatus back and forth is easier than rotating the apparatus, since it is possible that, with rotation of the hub, torque will not be transmitted to the distal end 114 of the catheter 112.

On the other hand, moving a relatively stiff tube with a pointed end in the heart, especially across the interatrial septum, may be risky in that the atrial wall could be perforated. To prevent this, the dilator/apparatus may be advanced and withdrawn over a guidewire that is, for example, between about 0.025 and about 0.032 inches in diameter. This guidewire may have a “preformed curve” at its distal end, as described previously. The guidewire 118, upon being advanced out of the dilator tip in the left atrium, will tend to coil 123 within the left atrial cavity (see FIG. 9). Thereafter, as the dilator 112 is being advanced back and forth across the PFO tunnel 124 during abrasion, the tip of the dilator 112 will be directed into the left atrial cavity along the guidewire 118 rather than towards the chamber wall. Thus, there is a reduced risk of perforating the chamber wall during the procedure.

In an alternative embodiment shown in FIG. 10, instead of a guidewire with a preformed curve, a deflectable or steerable catheter 212 may be used. In this embodiment, the tip of the catheter 212 may be advanced across the PFO into the left atrium. Such steerable or deflectable catheters may be directed in the desired direction using a variety of different methods/technologies to bend the catheter 212 tip—e.g., pullwire technology, use of a shape memory metal (e.g., Muscle Wire), or subjecting the catheter tip or the distal end 214 to a magnetic field (with a magnet located in the distal end 214 of the catheter 212). By using such steerable or deflectable catheters, the distal end 214 of the catheter 212 will be bent or deflected similar to manner in which the guidewire 118 is directed into the left atrial cavity as shown in FIG. 9. It is also contemplated that the dilator 212, with its abrading surface region 219, may be advanced back and forth across the PFO tunnel 224 over a steerable catheter (e.g., a steerable catheter having a 6 French to 8 French diameter), such that catheter 212 essentially replaces the guidewire 118 shown in FIG. 9. The tip or the distal end 214 of the catheter 212 will be bent in the left atrial cavity and allow the dilator 212 to be advanced back and forth across the tunnel 224 of the PFO.

The embodiment of FIG. 10 also includes an inflatable balloon 250 as the abrading surface region 219. This inflatable balloon 250 may be provided on a catheter 212 (e.g., a 6-9 French catheter), which may include one or more electrodes 216 at its distal end 214. These electrodes 216 may be used, for example, to record electrophysiological data and allow incorporation of the device into an electroanatomical navigation system. The catheter 212 may be steerable or deflectable using pullwire technology, or the use of a memory metal wire (e.g., Muscle Wire or Nitinol), or subjecting the catheter 212 to a magnetic field (with a magnetic sensor incorporated into the tip of the catheter 212) to selectively bend the distal tip to the desired radius of curvature. The inflatable balloon 250 may include a rough outer surface, such as by incorporating metal wires on its surface (see FIG. 10). Electrodes 116 may also be provided at the distal 214 and proximal 213 ends of the abrading surface region 219 (i.e., the balloon 250), thereby allowing visualization radiographically or via an electroanatomical mapping system. After the catheter 212 of FIG. 10 has been advanced across the PFO into the left atrial cavity, the distal end 214 of the catheter 212 will be deflected and the balloon 250 will be inflated to a predetermined pressure (it is contemplated that the balloon may be inflated with a radio-opaque fluid for purposes of visualization). Thereafter, the apparatus may be advanced back and forth across the PFO tunnel 224. At the end of the procedure, the balloon 250 is deflated and withdrawn into a sheath and the apparatus removed out of the body. It is also possible to use the catheter 212 to perform a “voltage map” of the PFO tunnel 224 to ensure that there is adequate electrophysiological evidence of injury and endothelial denudation.

In another embodiment of the invention, the septum primum and septum secundum can be injured (e.g., by promoting inflammation) by injecting a substance into the respective surfaces of either the septum primum or the septum secundum. This injected substance can be electroanatomically mapped. In one embodiment, the substance can include a biological material, such as, Freund's adjuvant, talc, GCSF (granulocyte colony stimulating factor), chemotactic factors, tumor necrosis factor, vascular endothelial growth factor, myoglobin, SDF1 or TXCR4, and echo or Coxsackie live attenuated virus.

The foregoing description of embodiments and examples of the invention has been presented for purposes of illustration and description. It is not intended to be exhaustive or limit the invention to the forms described. Numerous modifications are possible in light of the above teachings. Some of those modifications have been discussed, and others will be understood by those skilled in the art. The embodiments were chosen and described in order to best illustrate the principles of the invention and various embodiments as are suited to the particular use contemplated. It is hereby intended that the scope of the invention be defined by the claims appended hereto.

What is claimed is:

1. A method of closing a patent foramen ovale in a patient, comprising the steps of:
(a) locating a His bundle, plane of the interatrial septum, and coronary sinus ostium in a patient;
(b) identifying a fossa ovalis on the basis of one or more predetermined distances between said fossa ovalis and said His bundle, said plane of the interatrial septum, and said coronary sinus ostium;
(c) locating a patent foramen ovale by probing the junction between said fossa ovalis and a limbus of said fossa ovalis; and
(d) causing injury to the surfaces of at least one of a septum primum and a septum secundum within said patent foramen ovale.

2. The method of claim 1, wherein said step of causing injury comprises applying energy to said surfaces.

3. The method of claim 2, further comprising the step of monitoring electrical properties of said surfaces subsequent to said energy being applied to said surfaces.

4. The method of claim 2, wherein said energy comprises RF energy.

5. The method of claim 2, wherein said energy comprises non-RF energy.

6. The method of claim 1, wherein said step of causing injury comprises mechanically abrading said surfaces.

7. The method of claim 6, wherein said surfaces are mechanically abrading by an inflatable balloon.

8. The method of claim 1, wherein said step of causing injury comprises injecting a substance into said surfaces.

9. The method of claim 8, wherein said substances comprises a biological material.

10. The method of claim 1, wherein said patent foramen ovale and/or said fossa ovalis is located using an electroanatomical navigation system.

11. An apparatus for performing the method of claim 1 comprising:
(a) a catheter having one or more electrodes at the distal end thereof; and
(b) an electroanatomical navigation system having a display screen associated therewith.

12. The apparatus of claim 11, wherein said electroanatomical navigation system includes executable instructions for determining the location of said fossa ovalis and depicting a virtual fossa ovalis on said display screen in order to locate said patent foramen ovale and/or guide the application of energy to said surfaces of at least one of said septum primum and septum secundum within said patent foramen ovale.

13. The apparatus of claim 11, wherein the location of said fossa ovalis may be determined based on predetermined distances from structures in the heart and/or predetermined locations with respect to structures in the heart.

14. The apparatus of claim 11, wherein a surface of said catheter adjacent said distal end is configured for mechanical abrasion of said surfaces of at least one of said septum primum and said septum secundum within said patent foramen ovale.

15. A method of closing a patent foramen ovale in a patient, comprising the steps of:
(a) locating a tunnel of a patent foramen ovale by probing the junction between a fossa ovalis and a limbus of said fossa ovalis; and
(b) causing injury to the surfaces of at least one of a septum primum and a septum secundum within said tunnel of said patent foramen ovale by applying energy to at least one of said septum primum and said septum secundum.

16. The method of claim 15, wherein said energy comprises non-RF energy.

17. An apparatus for closing a patent foramen ovale in a patient, comprising:
(a) a catheter having one or more electrodes at the distal end thereof; and
(b) an abrading surface located proximate to said distal end of the catheter.

18. The apparatus of claim 17, wherein said electrodes are configured to emit RF, laser, microwave or ultrasonic energy.
